(19) United States
(12) Patent Application Publication
(10) Pub. No.: US 2025/0316350 A1
(43) Pub. Date: Oct. 9, 2025

(54) ASYNCHRONOUS DATA SHARING COMPUTING INFRASTRUCTURE

(71) Applicant: Friction Free Dental, Inc., Boston, MA (US)

(72) Inventor: Kevin Farrell, Boston, MA (US)

(21) Appl. No.: 19/172,846

(22) Filed: Apr. 8, 2025

Related U.S. Application Data
(60) Provisional application No. 63/631,604, filed on Apr. 9, 2024.

(51) Int. Cl. G16H 10/60

(52) U.S. Cl. CPC G16H 10/60

(57) ABSTRACT

A system and method for providing asynchronous data collaboration. A disclosed system includes: data handling services for reading and writing data to and from a plurality of legacy information systems and for transforming and storing data in a universal data record (UDR) format; universal display interface services configured for displaying data in the UDR format to a set of clients; collaboration services configured for creating and sharing active capture recordings; and secure script artificial intelligence (S2AI) services configured to securely transcribe, store and enhance natural language inputs.

### PRIORITY CLAIM

This application claims priority to co-pending provisional patent application Ser. No. 63/631,604 filed on Apr. 9, 2024, entitled ASYNCHRONOUS DATA SHARING COMPUTING INFRASTRUCTURE, the contents of which are hereby incorporated by reference.

### TECHNICAL FIELD

The subject matter of this disclosure relates to a computing infrastructure that supports asynchronous data sharing, and more particularly to a cloud services infrastructure that supports information collaboration among platforms by using and normalizing data from disparate information systems.

### BACKGROUND

There exists any number of domains in which people need to communicate, share information and collaborate to more efficiently and effectively achieve a goal or desired outcome. Such collaboration is often enhanced when visual or graphical information can be shared. Illustrative domains where sharing such information may for example include medical, engineering, software development, marketing/advertising, manufacturing, legal, etc. However, relevant information is often stored in legacy disparate or proprietary information processing systems that are not designed to share data or interact with each other. Accordingly, collaboration is limited or not possible, burdening the people and entire domain, every day, with unnecessary slow, painstaking, and costly processes.

### SUMMARY

Aspects of the disclosure provide a system and method for providing asynchronous data collaboration.

In one aspect, a disclosed system is provided that includes a memory and a processor coupled to the memory and configured to provide a data collaboration platform, the platform having: a data handling service for reading legacy data from and writing legacy data to a plurality of disparate information systems and for transforming and processing legacy data using a common universal data record (UDR) format; a universal display interface (UDI) service configured to: display a UDI at remote clients, wherein the UDI includes a primary interactive diagram; and overlay case-specific graphical information onto the primary interactive diagram, wherein the graphical information is rendered from data obtained from at least one disparate information system; and a collaboration service configured to: create active capture recordings from within the UDI at the remote clients, wherein the active capture recordings include screen activity, audio, and video footage; and share active capture recordings with other remote clients in a secure collaboration interface within the UDI.

A further aspects provides a method, comprising: rendering a universal display interface (UDI) at a remote client, wherein the UDI includes a primary interactive diagram; reading legacy data from at least one of a plurality of disparate information systems and transforming the legacy data into a common universal data record (UDR) format; overlaying patient specific graphical information onto the primary interactive diagram, wherein the graphical information is rendered from legacy data obtained from the at least one disparate information system; and providing a facility for creating an active capture recording from within the UDI, wherein the active capture recording includes screen activity, audio, and video footage; and providing a collaboration interface within the UDI, and securely sharing a collaboration message with a second remote client, wherein the collaboration message includes the active capture recording.

Another aspect of the disclosure includes any of the aspects, and wherein reading legacy data from and writing legacy data to the plurality of disparate information systems utilizes robotic process automation to interact with user interfaces of the plurality of disparate information systems.

Another aspect of the disclosure includes any of the aspects, and wherein reading legacy data from and writing legacy data to the plurality of disparate information systems utilizes application programming interfaces.

Another aspect of the disclosure includes any of the aspects, and wherein the plurality of disparate information systems utilize different commands and file systems to access and store data.

Another aspect of the disclosure includes any of the aspects, and wherein the UDR format includes a system agnostic file type for similar types of data.

Another aspect of the disclosure includes any of the aspects, and wherein the UDR format further includes a system agnostic naming convention.

Another aspect of the disclosure includes any of the aspects, and wherein the legacy data includes dental image data and dental text data.

Another aspect of the disclosure includes any of the aspects, and wherein the primary interactive diagram includes a generic chart of a set of teeth.

Another aspect of the disclosure includes any of the aspects, and wherein the overlay depicts patient specific dental work rendered on the set of teeth.

Another aspect of the disclosure includes any of the aspects, and wherein the UDI further includes a media file viewer for viewing dental images of a selected patient.

Another aspect of the disclosure includes any of the aspects, and wherein the UDI further includes a notes interface for viewing and creating patient specific notes.

Another aspect of the disclosure includes any of the aspects, and further comprising a secure script artificial intelligence (S2AI) service configured to securely transcribe and store natural language inputs within the notes interface.

Another aspect of the disclosure includes any of the aspects, and wherein the S2AI service includes a generative AI enhancement service that processes natural language inputs.

Another aspect of the disclosure includes any of the aspects, and wherein the generative AI enhancement service converts natural language inputs into formatted medical notes.

Another aspect of the disclosure includes any of the aspects, and wherein the collaboration interface includes a facility for a first user to create a collaboration with a second user and a content window for viewing and sharing messages with the second user.

Another aspect of the disclosure includes any of the aspects, and wherein the content widow is configured to send and display active capture recordings.

Another aspect of the disclosure includes any of the aspects, and wherein the collaboration service includes annotation tools for annotating displays within the UDI.

Another aspect of the disclosure includes any of the aspects, and wherein the UDI further includes a secondary interactive diagram that comprises a generic periodontic chart of a set of teeth.

Another aspect of the disclosure includes any of the aspects, and wherein the UDI service is configured to overlay patient specific periodontic information onto the secondary interactive diagram, wherein the graphical information is rendered from data obtained from at least one disparate information system.

Two or more aspects described in this disclosure, including those described in this summary section, may be combined to form implementations not specifically described herein. That is, all embodiments described herein can be combined with each other.

The details of one or more implementations are set forth in the accompanying drawings and the description below. Other features, objects and advantages will be apparent from the description and drawings, and from the claims.

### BRIEF DESCRIPTION OF THE DRAWINGS

These and other features of this invention will be more readily understood from the following detailed description of the various aspects of the invention taken in conjunction with the accompanying drawings in which:

FIG. 1 shows a collaborative computing infrastructure according to embodiments.

FIG. 2 depicts Secure Script AI (S2AI) Services according to embodiments.

FIG. 3 shows an XDAT client according to embodiments.

FIG. 4 shows a primary interactive diagram according to embodiments.

FIG. 5 shows a further view of a primary interactive diagram according to embodiments.

FIG. 6 shows a media file viewer according to embodiments.

FIG. 7 shows a notes interface according to embodiments.

FIG. 8 depicts a secondary interactive diagram according to embodiments.

FIG. 9 depicts an active capture process according to embodiments.

FIG. 10 depicts a collaboration interface according to embodiments.

FIG. 11 depicts a playback tool according to embodiments.

FIG. 12 depicts an XDAT cloud/client architecture according to embodiments.

FIG. 13 shows a network according to embodiments.

FIG. 14 shows a computing system according to embodiments.

The drawings are not necessarily to scale. The drawings are merely schematic representations, not intended to portray specific parameters of the invention. The drawings are intended to depict only typical embodiments of the invention, and therefore should not be considered as limiting the scope of the invention. In the drawings, like numbering represents like elements.

### DETAILED DESCRIPTION

Various embodiments provided herein relate to a computing infrastructure for uniformly viewing and sharing information amongst users that have disparate information systems. Disparate information systems for example comprise any type of information technology platform that utilize different commands, file systems, and/or processes to access and store data. In certain aspects, information in the present approach is presented in an interactive and graphical platform that displays data in a format independent of the underlying information systems from which data is collected. Additionally, interactions with the graphical information (e.g., voice and video inputs, annotations, navigation, etc.) by a first user can be captured (i.e., recorded) and forwarded to a second user for playback in a secure manner. Interactions by the second user can likewise be captured and sent back to the first user in a secure manner, thus providing a system agnostic asynchronous collaboration platform. This approach provides a highly effective and efficient digital platform to create/capture and playback such collaborations asynchronously, by enabling users to easily speak and interact with content as they would in-person thus allowing for a more efficient productive use of participants' time who no longer need to coordinate synchronous collaborations together. Collectively, the ability to capture, playback and communicate asynchronously, is herein referenced as eXtreme Digital Articulation Technology (XDAT).

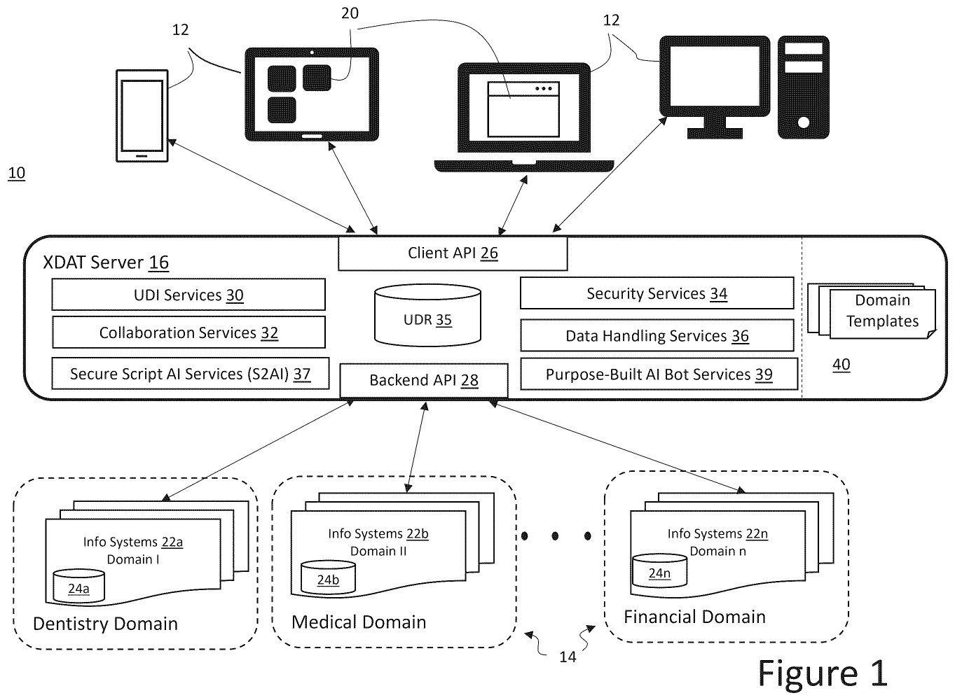

Referring to FIG. 1, an illustrative collaborative computing infrastructure 10 is depicted that allows for the asynchronous sharing of information amongst users operating within a common domain. Infrastructure 10 is managed by an XDAT server 16, which may for example be implemented as a software as a service (SaaS) network, cloud, and/or other system(s). XDAT server 16 includes a number of services that allow users operating client devices 12 to collaborate within a given domain and across domains 14. Domains 14 can, e.g., be instantiated by any group of users (within some field of endeavor) that need to share sensitive information, i.e., system data from databases (e.g., 24a) in their domain. A typical domain includes a number of information systems (e.g., 22a) that include legacy data and are generally private and closed off to other domain users. XDAT server 16 may be configured to implement any number of separate and discrete domains 14. Such domains 14 may for example comprise a dentistry domain, a medical domain, a financial domain, software development domain, an insurance domain, a legal domain, etc.

As shown, each domain 14 may include any number of legacy information systems active within the domain, and each system generally comprises a closed database only accessible by a particular business or enterprise. XDAT server 16 provides a secure platform that allows users to seamlessly share legacy data and collaborate within their domain. In a dentistry domain for example, a dentist may need to share data from the dentist's private practice management system and collaborate with an endodontist that does not have access to the dentist's practice management system. XDAT server 16 provides a set of services that allows participants to view and share private legacy data in a system agnostic manner. As shown, each information system is connected to the XDAT server 16 via a backend API 28, that allows legacy data from each system to be read or written in a real-time, as needed manner.

XDAT server 16 accordingly allows users to collaborate and share such legacy data from information systems via client devices 12 that connect to the XDAT server 16 via a client API 26. Client devices 12 may comprise any type of computing device, e.g., a smartphone, a tablet, a laptop, a desktop, etc. Collaboration and sharing on a client device 12 may occur via an XDAT client 20 that may include a downloadable application, a browser, or any other interactive technology.

As noted, XDAT server 16 includes a number of services that allow for collaboration and sharing. Some of the core services include: a universal display interface (UDI) service 30, collaboration service 32, security service 34, data handling service 36, Secure Script AI (S2AI) service 37 and purpose-built AI Bot (PAIB) service 39.

Universal Display Interface (UDI) service 30 provides an agnostic platform for interactively displaying legacy data from different information systems within a domain. Information from the information systems (e.g., 22a) is communicated back and forth with XDAT server 16 via backend API 28 by data handling service 36, and data within the XDAT server 16 is transformed and temporally stored as Universal Data Records 35 (UDRs).

UDRs provide normalized records of data that can be easily and consistently processed by UDI services 30. For example, medical notes from a first information system may be stored as PDF records, while medical notes from a second information system may be stored as XML records. The PDF and XML records when read into the XDAT server are transformed into UDRs, which is a common system agnostic format. Furthermore, disparate information systems 22a may store structured data in different arrangements, e.g., system 1 may store a note as <title><date><summary><conclusion> while system 2 stores a note as <date><title><conclusion><summary>. Data handling service 36 automatically transforms such data into a system agnostic UDR having a common structured format. Similarly, unstructured data and media files will likewise be transformed into common UDR formats, e.g., common naming schemes, wrappers, file formats, etc. In certain cases, the UDR format includes a system agnostic file type and a system agnostic naming convention for different types of data (e.g., notes files are transformed into notes UDRs, patient details are transformed into patient UDRs, etc.).

Collaboration service 32 provides a platform for capturing and sharing messages and mixed media information with other users within a domain. Security service 34 provides a platform for controlling system and data access to information from the various users of client devices 12 within a domain. As noted, data handling service 36 provides a data processing platform for reading and writing data from the information systems within a domain. XDAT server 16 may also provide additional services and sub-services, some of which are described herein. Additionally, as shown, new or existing domains may be implemented using a domain template 40 that provides an initial configuration of XDAT services.

Secure Script AI service (S2AI) 37 provides secure natural language processing services, e.g., allowing users to share and speak to sensitive private data and take advantage of generative AI models (e.g., XDAT GPT capabilities), all within a closed secure system allowing users to leverage the power of AI with the peace of mind knowing their data will remain confidential. In contrast to open models, e.g., Apple Siri, Google Assistant, Amazon Alexa, and the like, where similar private data is not secured, S2AI 37 is purpose-built to ensure any user content and data is kept private and contained to the XDAT system. For example, a dentist could dictate a patient's private medical record and findings for a clinical note, whereby S2AI 37 would utilize its closed services to store original audio, transcribe and enhance transcriptions using S2AI GPT.

As shown in FIG. 1, each information system (e.g., 22a) within a domain generally includes multiple unique and proprietary system databases (e.g., 24a) tailored for a particular application (e.g., a practice management system, an image database, a parts database, a media database, a docketing system, etc.). The collection of databases within a domain are also referred to herein as “system databases.” In some instances, a business or enterprise may have more than one information system. In other cases, different businesses or enterprises may have different types of information systems that perform similar or different functions. The type and number of information systems within a domain will depend on the number of participants and the particular domain in which the infrastructure 10 is utilized. For example, in a medical domain, a first legacy system may include patient records, a second legacy system may include image data, a third legacy system may include billing information, etc. In a marketing/advertising production domain, a first legacy system may include storyboards, a second legacy system may include stock image data, a third legacy system may include music, etc. In an engineering domain such an oil exploration, a first legacy system may include 3D maps, a second legacy system may include geology data, a third legacy system may include geopolitical data, etc. In a computer science domain, a first legacy system may include source control, a second legacy system may include design and User Interface (UI), a third legacy system may include data in a database and corresponding schema, etc. As noted, legacy systems within a domain may differ amongst different businesses or enterprises. For example, in a medical domain, a first business may use a first type of practice management system while a second business may use a second type of practice management system.

As noted, in certain cases, data is accessed and written to information systems using a backend API 28. However, certain information systems have no or a limited API interface to access data. In such cases, the XDAT Server 16 can utilize a Purpose-Built AI BOT (PAIB) service 39, e.g., leveraging an RPA (Robotic Process Automation) approach to directly interact with one or more information system user interfaces (UI), to perform operations directly by automatically navigating through the application, to extract, save or update data from/to the information system. RPA uses a set of instructions for the bot to mimic human-computer interactions (e.g., keystrokes and mouse actions) to carry out tasks such as accessing and storing data and files on legacy information systems. Such interactions may include performing specific actions (select, click, copy, cut-and-paste, move, etc.) that are otherwise performed manually to complete the tasks.

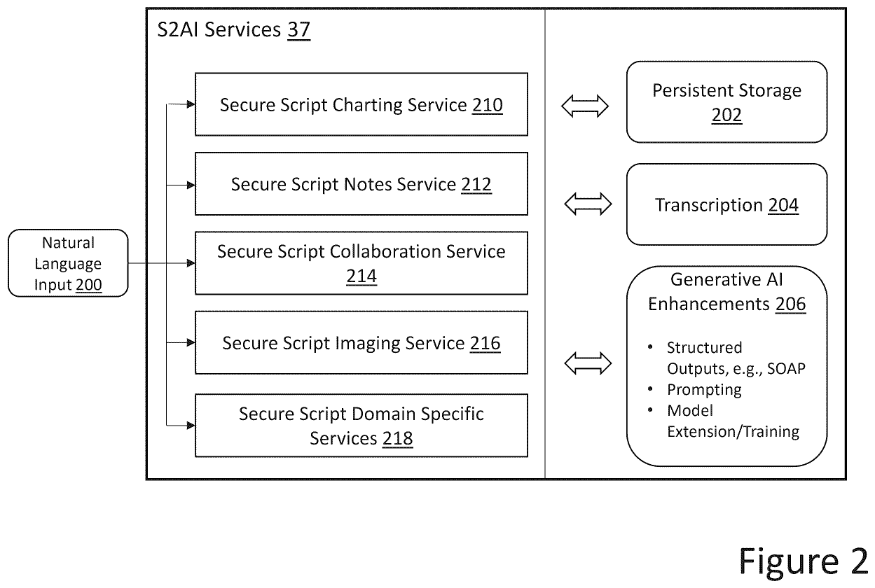

FIG. 2 depicts an illustrative implementation of various S2AI services 37. These various services integrate with the features described herein to process natural language (NL) inputs 200 in a closed and secure environment. In particular, captured NL inputs 200 can be stored in a persistent storage 202 to allow a user to later hear the original input. Additionally, NL inputs 200 can be transcribed with a transcription system 204 into text. Further, NL inputs 200 can be processed by a set of generative AI enhancements 206, such as converting unstructured text into structured outputs, e.g., SOAP, structured reports, etc., service specific prompting can be deployed to augment or enhance the outputs, and model extension/training may be utilized to provide secure and specialized outputs. S2AI services 37 can for example be incorporated into a secure script charting service 210, a secure script notes service 212, a secure script collaboration service 214, a secure script imaging service 216 and/or secure script domain specific services.

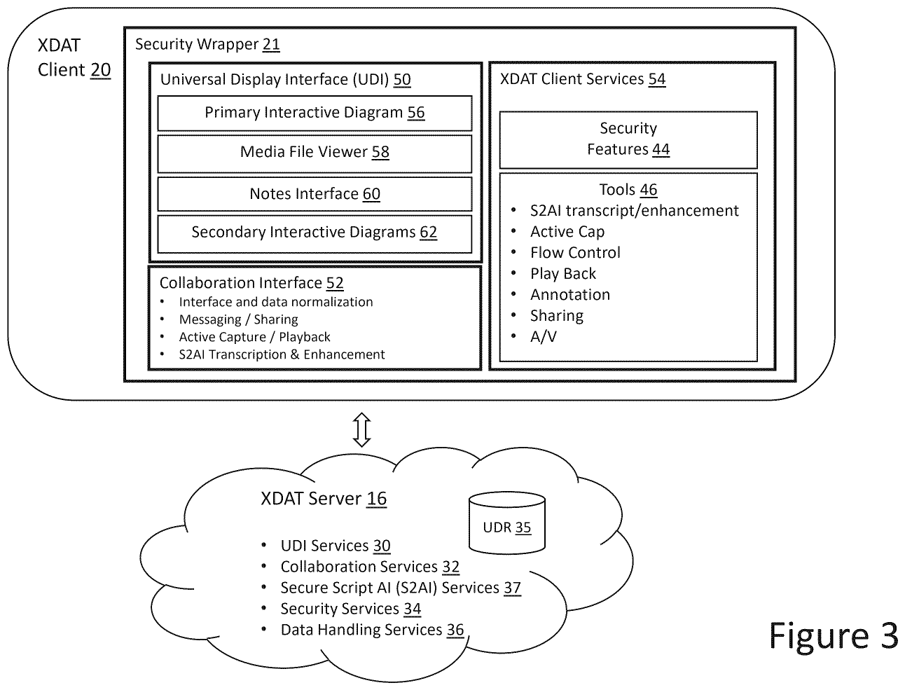

Within each domain, one or more XDAT clients 20 are configured to access and process data from system databases (e.g., 24a) within information systems 22a. FIG. 3 depicts illustrative functionality provided within an XDAT client 20, which may be implemented in a lightweight manner such that most of the processing is done at the XDAT server 16, e.g., in certain embodiments XDAT client 20 is a virtual representation controlled and generated by XDAT server 16. As shown, XDAT client 20 includes a security wrapper 21 that implements a number of security features 44 (e.g., login, authentication, access control, etc.) that control the type and amount of information each user is able to access via a client 20. The security wrapper 21 is for example implemented and controlled by local security features 44 and security services 34 provided by the XDAT server 16.

In this example, XDAT client 20 generally includes: (1) a universal display interface (UDI) 50 that allows a user to display and interact with normalized legacy data, i.e., Universal Data Records (UDRs) in a system agnostic environment; (2) a collaboration interface 52 that allows users to create, share and receive information with other users; and (3) a set of XDAT client services 54 configured to support operations within the XDAT client 20.

As noted, universal display interface (UDI) 50 allows for user interaction with legacy data that is captured from the underlying information systems. The format and presentation of UDI 50 is implemented by UDI services 30 in conjunction with the UDRs 35 and will largely depend on the particular domain the user is operating within. In certain embodiments, system data (e.g., a patient record, a project, etc.) is processed and graphically displayed within one or more interactive diagrams. Users can interact with a diagram to view or navigate to more detailed information or interfaces. Data can also be collected from a user within the universal display interface 30 and stored back in information databases in a seamless manner.

Collaboration interface 52 provides a platform in which system data and user analysis (e.g., navigating through UDI screens, annotations, etc.) can be integrated and shared with other users. Within collaboration interface 52, users can also actively capture interactions performed within (or outside) universal display interface 50, including screen recording of any application on the device that is displayed to a user, again with annotations, voice inputs, webcam video inputs, etc. The recording of such information, referred to herein as an “active capture,” can then be shared with other users via collaboration interface 52 and collaboration services provided by the XDAT cloud 16. In addition, the collaboration interface 52 also provides S2AI transcription and enhancement services, whereby, e.g., users can playback in silence for privacy and all active captures are automatically transcribed and can be viewed with sub-titles displaying the transcription.

In the illustrative embodiment shown, universal display interface 50 includes a primary interactive diagram 56 that is presented to the user and displays a domain specific interactive graphical image, onto which processed case-specific data is automatically overlayed. For example, in a dentistry domain, the graphical image might include a generic chart of a set of teeth onto which image data (e.g., cavity locations, filling types, crowns, etc.) associated with a selected patient is overlayed. In an advertising/marketing domain, the graphical image might include a generic storyboard, onto which system data associated with a selected project is overlayed. In an oil exploration domain, the graphical image might include a generic earth cross-section, onto which geological data associated with different earth layers is overlayed.

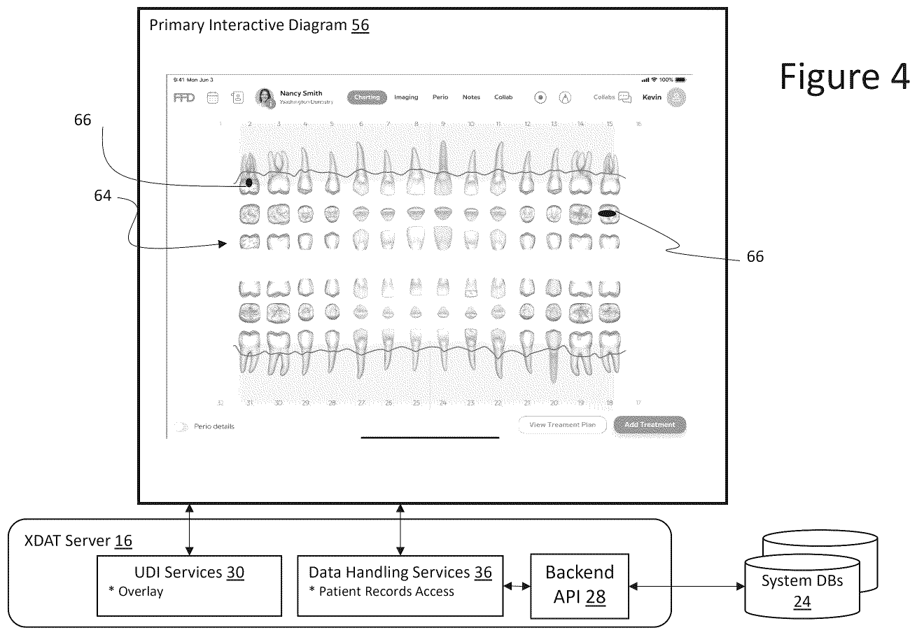

FIG. 4 depicts an illustrative primary interactive diagram 56 for a dentistry domain example. In this case, diagram 56 includes a generic graphical image 64 (i.e., a chart or rendering of a set of teeth) and patient specific image data 66 (e.g., locations of prior dental work) overlayed on the graphical image 64. Additionally, color coded treatment plans may be included and distinguished from existing work, e.g., plans in red, existing completed work in green, etc. Patient specific image data 66 is for example obtained via a patient record access service that is implemented by data handling services 36, which makes a call to backend API 28 to retrieve records from one or more system databases 24. The overlay process is implemented by UDI services 30, which processes the retrieved data and creates and renders the patient specific image data 66 (e.g., tooth number, treatment type, treatment type attributes, with different colors indicating planned/red versus existing/green, etc.) on the graphical image 64.

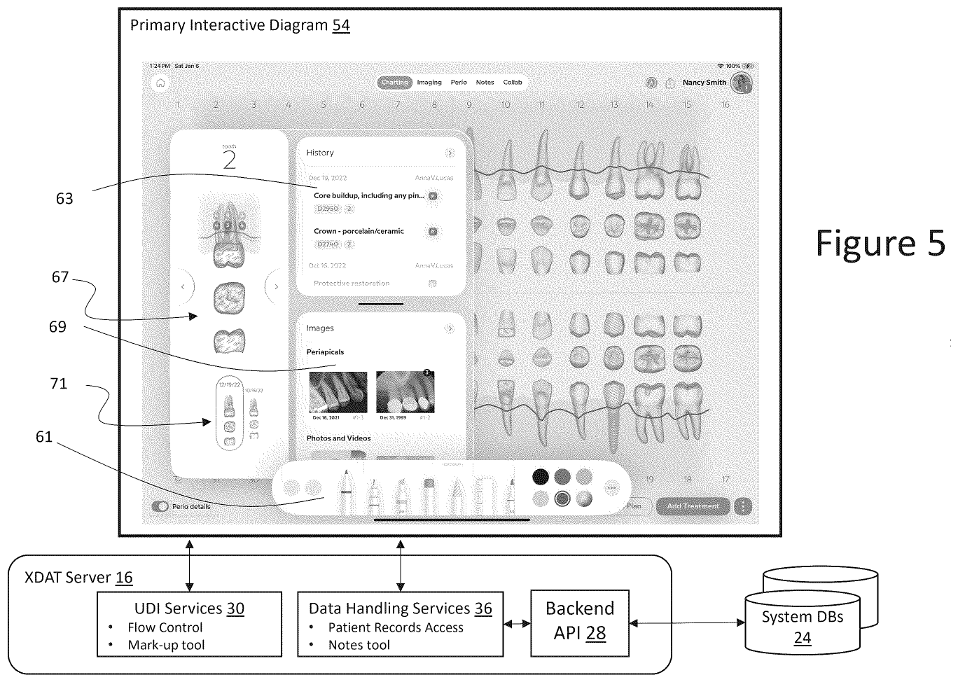

Within the primary interactive diagram 56, the user is able to select and interact with various elements of the image 64. For example, in this dentistry domain case, the user is able to select a tooth (e.g., with a touch screen or mouse action) and navigate to more detailed images and data. An example of this is shown in FIG. 5, in which more granular details of a particular tooth are presented in new dialog windows 67, 69 in response to such a selection. When this occurs, the data handling service 36 and backend API 28 make several different calls to the system DBs 24, aggregating all the information shown in a single consolidated screen 67. In addition, within screen 67, a history selector 71 allows for the full display to repopulate according to a selected date or appointment to generate and show what the selected tooth state looked like at any given appointment. For example, a dentist may be interested when a tooth was extracted and likewise, what the condition of the tooth was leading up to and need for the tooth to be extracted. This history is being automatically generated by the XDAT server from a visit ledger and history.

The capabilities to navigate and interact with diagram 54 is determined by a flow control service, implemented by UDI services 30. Flow control service dictates the flow paths, windows, screens, data, etc., available to the user and can for example be preconfigured and tailored for a particular domain. Tools 46 (FIG. 3) provide the various tools that the user is able to utilize to view, process and enhance data. For example, as shown in FIG. 5, a mark-up tool 61 is provided for marking up and annotating screen images. Also shown is a notes tool 63 for viewing and inputting notations for the selected tooth into a system database 24.

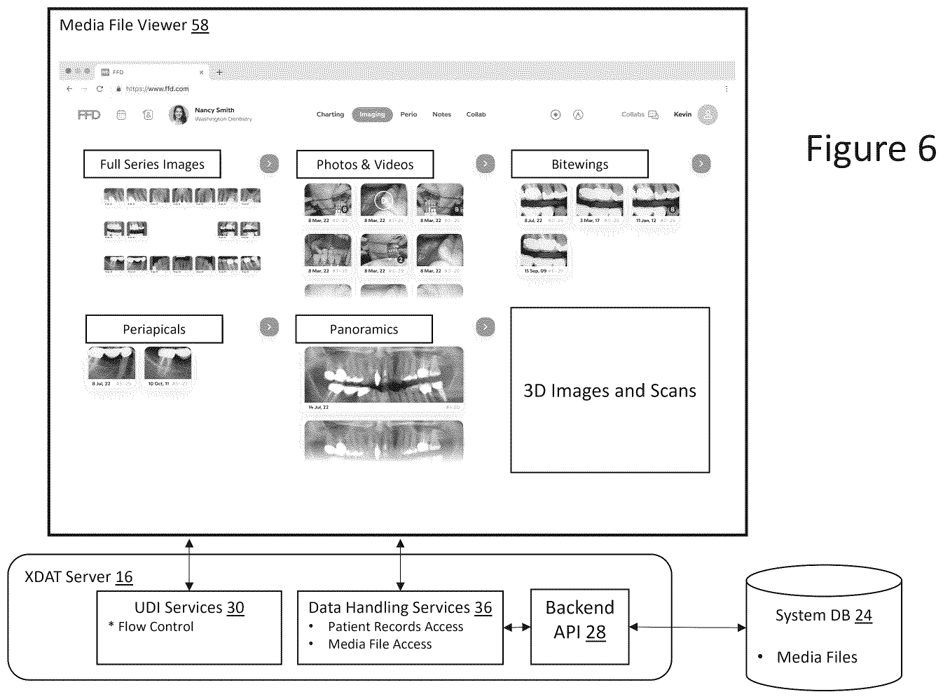

FIG. 6 depicts an illustration of a media file viewer 58, which allows the user to view and interact with media files from one or more system databases 24 containing media files, e.g., JPEGs, video files, audio files, 3D files (i.e., MRI), etc. In a typical domain infrastructure, media files often reside in a separate information system 22 (e.g., in an MRI database, various different independent imaging equipment databases, photos stored on a memory card on a SLR camara, a photo library on smartphone, etc.). Media file viewer 58 accordingly allows such media data to be presented and shown in a single aggregate view in the universal display interface 50, independent of the source or location of the disparate intendent databases where the images are stored. Data handling services 36 and backend API 28 manage the collection of the various images from their respective disparate sources and aggregate the view for presentation in 58.

In the dental example shown, media files from different sources are automatically categorized as full series images, photos & videos, bitewings, periapicals, panoramics, and 3D images and scans.

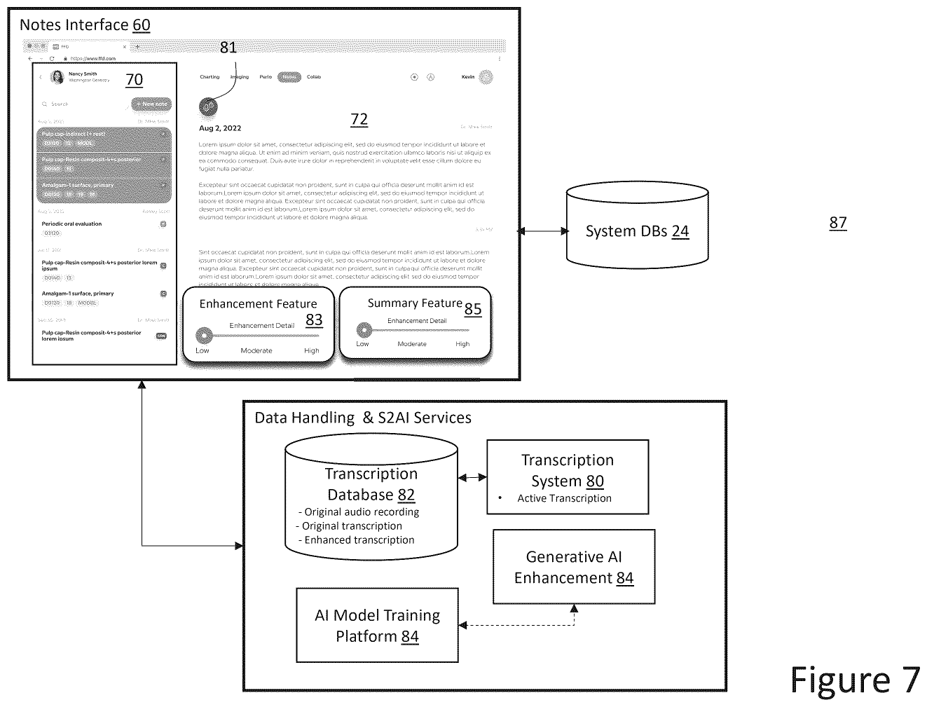

FIG. 7 depict an illustrative notes interface 60, that includes a first window 70 that displays a notes history for a selected patient and a notes window 72 that displays a selected note. Notes may include any information that the user wants to view or create regarding a presented data record (e.g., in the context of dental, patient feedback, patient findings, patient treatment plans, patient work completed that day, etc.). Window 70 depicts a screen in which the user can review existing notes from one or more system databases 24 and window 72 depicts a screen in which the user can create or enhance notes directly or through voice transcription using S2AI services and store notes in system database(s) 24. Accordingly, notes from one or more information systems 22 (FIG. 1) can be viewed, edited, created and saved. Notes, such as medical notes regarding a patient, can be generated either by typing or via a voice transcription system 80 that generates text based on voice input. In various aspects, a transcription database 82 (e.g., stored in XDAT cloud 16) may be utilized to store the voice/audio input, e.g., as a redundancy to the generated text. Furthermore and specific to S2AI enhancement, cryptic or technical notes written or otherwise dictated and transcribed in S2AI first means that original audio recording of what was said is always saved along with the original transcription to the DB 82.

A generative AI enhancement system 84 may also be provided to enhance or summarize the displayed note. For example, generative AI enhancement 84 is available to augment or transform the transcription into paragraph, bullet, or an industry-specific standard, for example into a medical SOAP (Subjective, Objective, Assessment, Plan) format. Window 72 further includes an S2AI enhancement button 81 that provides an enhancement feature 83 and a summary feature, which for example include a slider that allows user adjustment of the AI enhancement or summarization. Namely, the S2AI service can be adjusted to include more (verbose) or less (concise) output based on a selected level in the interface 60. For example, in a dental or medical application, a doctor can transcribe patient details which can then be enhanced and transformed into a medical standard SOAP (Subjective, Objective, Assessment, Plan) format, along with the original transcription and audio being saved for later reference. When button 81 is selected, enhancement and summary options 83, 85 may be displayed, allowing the user to select or adjust the enhancement or summary, e.g., with a slide bar 87. For example, an entire exam can be recorded start to finish in a note and S2AI can then provide a concise summary 85 of the note using generative AI enhancement 84, e.g., in paragraph, bullet or SOAP format.

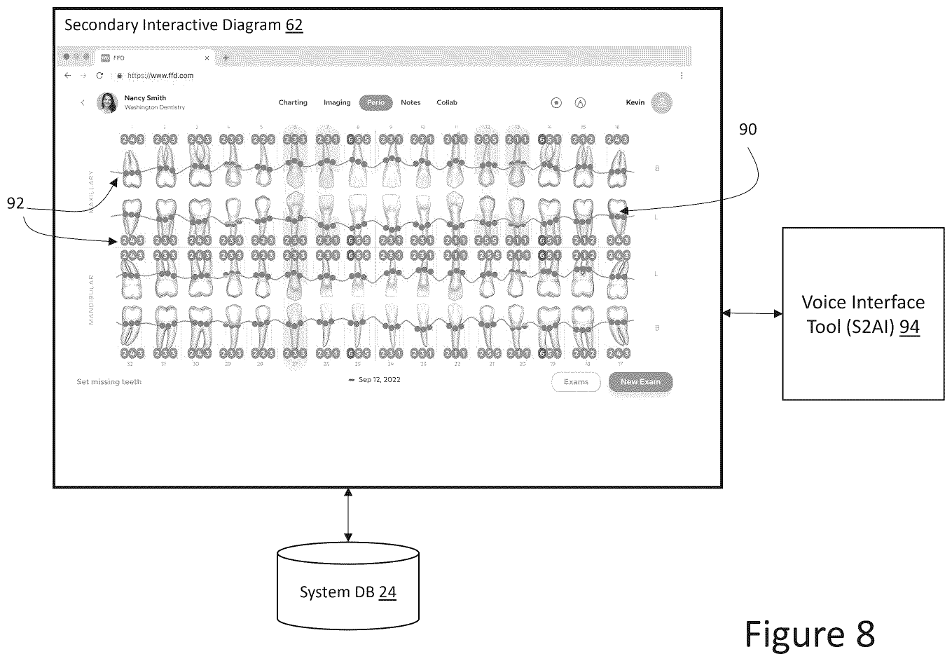

FIG. 8 depicts an illustrative secondary interactive diagram 62. In this case, diagram 40 depicts periodontics data 92 (i.e., gum health) for a selected patient record overlayed on top of an interactive graphic tooth image 90. Secondary interactive diagram 40 operates similarly to the primary interactive diagram 34, but depicts a different set of graphical information within the domain. Furthermore, in this embodiment, a voice interface tool 94 (that incorporates S2AI) is also provided that allows a user to make data entries (e.g., gum measurements) using voice input that will appear in the diagram 62 and be saved to a system database 24.

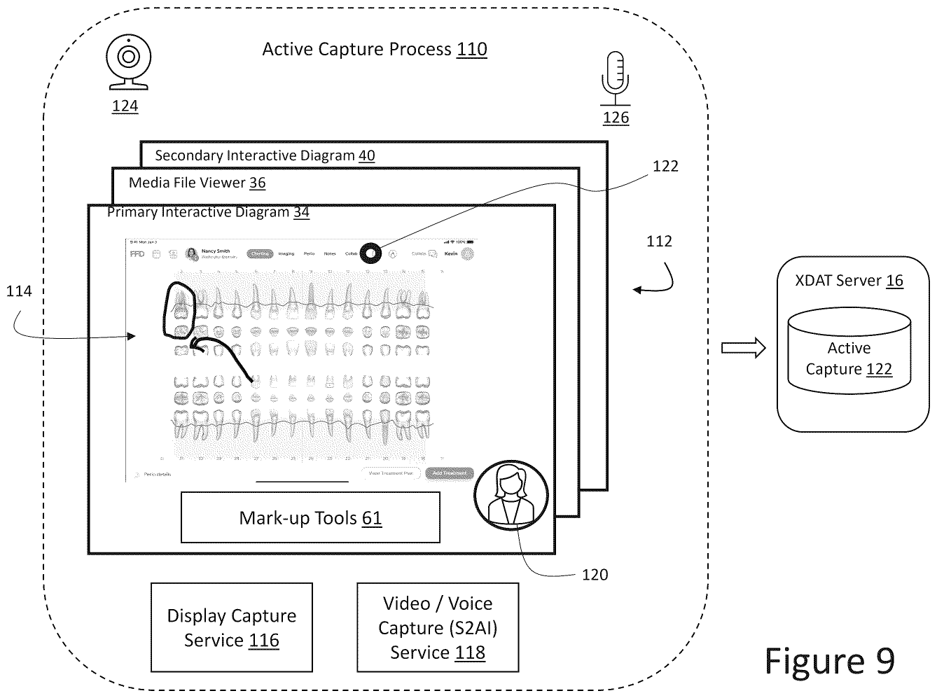

FIG. 9 depicts an example of an active capture process 110. The active capture process 110 allows a user to capture their actions while interacting within the XDAT client/device (as well as interactions outside an XDAT client). In this example, the process 110 includes: (1) a display capture service 116 that will capture and record what is being viewed on the user's device screen; and (2) a video/voice capture service 118 (incorporating SA2I) that will capture and record both video and audio of the user, e.g., via a webcam. The resulting information is packaged, transcribed (e.g., in subtitles) and outputted as an active capture 122, that can be stored or shared.

In this example, the user launches an active capture process 110 (e.g., by clicking a record button 122). As the user navigates through various screens and windows 112, display capture service 116 records the interaction as a streaming video. Additionally, a set of mark-up tools 61 (e.g., paint brushes, pencils, markers, shapes, erasures, colors, etc.) are provided that allows the user to markup what is being displayed, which is also captured by display capture service 116. At the same time, video of the user is captured by webcam 124 and displayed in window 120, and audio of the user is captured by microphone 126 (by video/voice capture service 118). The resulting captured information is packaged into an audio/video (AV) file that is outputted as active capture 122 to the XDAT server 16. Accordingly, when the active capture 122 is played back, it will show the screens 112 as the user viewed them, annotations the user made, as well as the user's audio and video inputs.

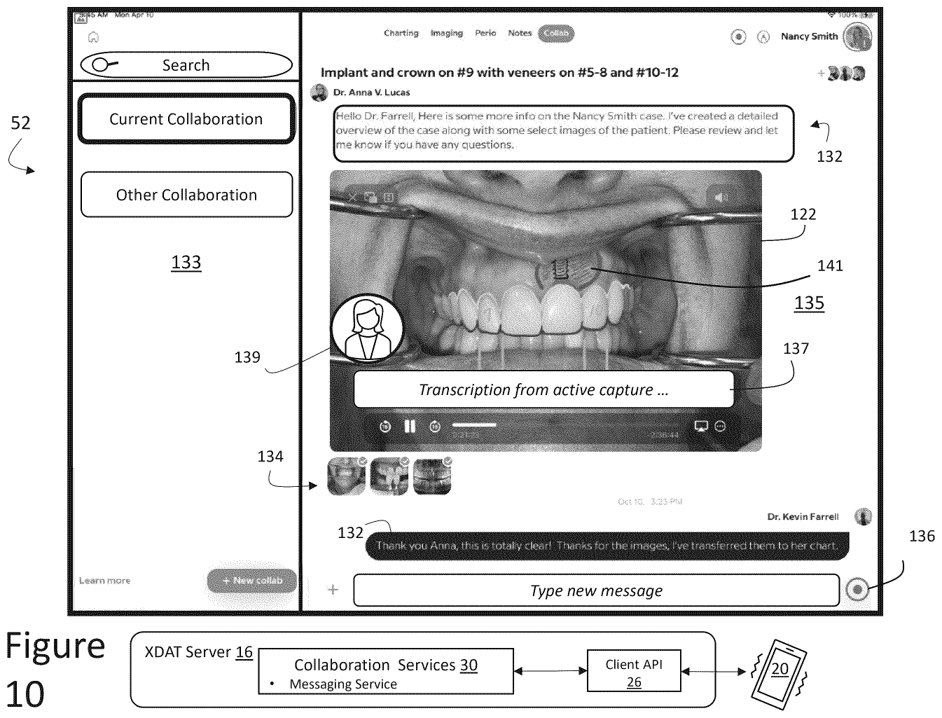

FIG. 10 depicts a collaboration interface 52 as viewed at a client device 20. A collaborations overview panel 133 provides an area where the user can search and select a current collaboration (e.g., a group chat between the user and one or more other users), or create a new collaboration. Once a collaboration is selected, the associated collaboration content is displayed in content window 135. From the content window 135, the user can create, view and respond to messages 132, create or view attachments 134 (e.g., media files) and/or playback an active capture 122 created by another user in the current collaboration.

In this example, an active capture 122 was included, which further depicts various features of the active capture 122. In the active capture 122 shown, a transcription of the associated audio is automatically provided in subtitles as well as video 139 of the sending user captured during the recording of the active capture. Further, markings 141 made with a markup tool are shown. The receiving user can also initiate a responsive active capture 136, in which the receiving user can record their own active capture, e.g., containing audio, video, screen displays, annotations, etc. The resulting responsive active capture can then be messaged back to the other users in the current collaboration.

When a new collaboration is created and a message is composed, the message can be sent via a messaging service within collaboration service 30. In the case where a selected user has an established account within the domain, a notification will be sent and the receiving user will receive a notification within their client 20. In the case where the selected user does not have an established account or appropriate security level, an email or SMS message containing a link will be sent to the selected user, which will provide the receiving user options for viewing the message if permitted.

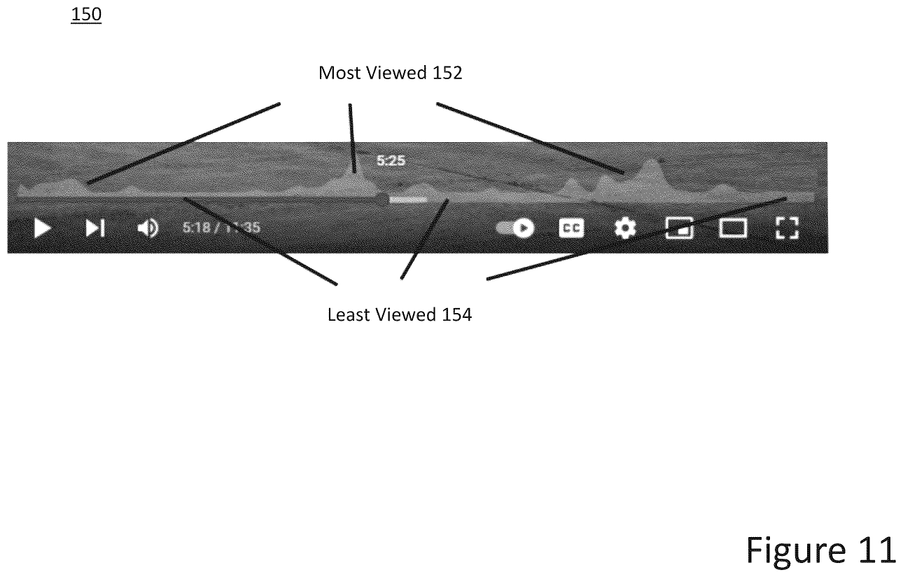

FIG. 11 depicts an illustrative active capture playback tool 150. The tool 150 includes a graphical summary of the most viewed 152 and least viewed 154 parts of an active capture. This feature allows, e.g., a dentist, to more quickly navigate to relevant parts of the active capture.

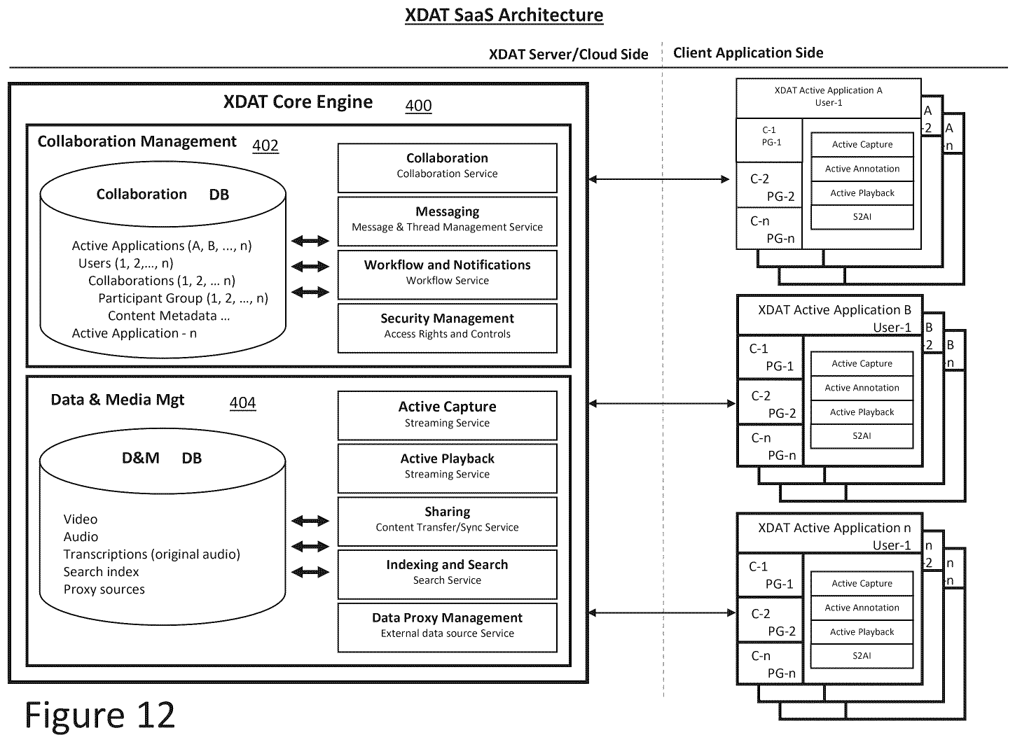

FIG. 12 depicts an illustrative XDAT SaaS architecture. XDAT server is for example implemented by an XDAT core engine 400 that for example includes collaboration management 402 and data & media (D&M) management 404. Collaboration management 402 includes a collaboration database that stores data associated with users, collaborations, etc., as well as various services for handling collaborations. D&M management 404 similarly include a D&M database that stores data and media obtained from system databases, as well as various services for handling data.

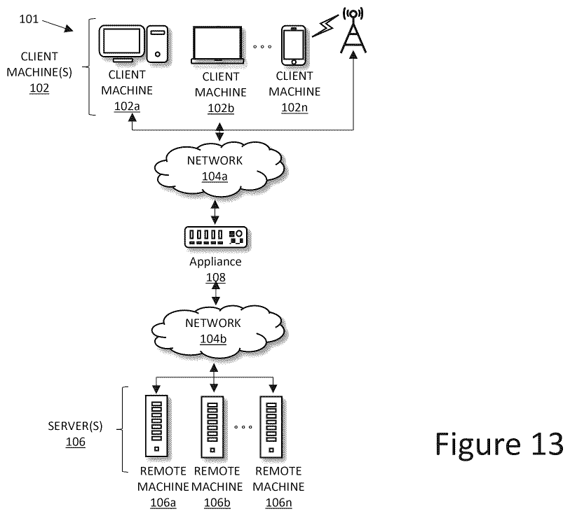

It is understood that aspects of the described infrastructure can be implemented in any manner, e.g., as a stand-alone system, a distributed system, within a network environment, etc. Referring to FIG. 13, a non-limiting network environment 101 in which various aspects of the disclosure may be implemented includes one or more client machines 102A-102N, one or more remote machines 106A-106N, one or more networks 104, 104′, and one or more appliances 108 installed within the computing environment 101. The client machines 102A-102N communicate with the remote machines 106A-106N via the networks 104, 104′.

In some embodiments, the client machines 102A-102N communicate with the remote machines 106A-106N via an intermediary appliance 108. The illustrated appliance 108 is positioned between the networks 104, 104′ and may also be referred to as a network interface or gateway. In some embodiments, the appliance 108 may operate as an application delivery controller (ADC) to provide clients with access to business applications and other data deployed in a datacenter, the cloud, or delivered as Software as a Service (SaaS) across a range of client devices, and/or provide other functionality such as load balancing, etc. In some embodiments, multiple appliances 108 may be used, and the appliance(s) 108 may be deployed as part of the network 104 and/or 104′.

The client machines 102A-102N may be generally referred to as client machines 102, local machines 102, clients 102, client nodes 102, client computers 102, client devices 102, computing devices 102, endpoints 102, or endpoint nodes 102. The remote machines 106A-106N may be generally referred to as servers 106 or a server farm 106. In some embodiments, a client device 102 may have the capacity to function as both a client node seeking access to resources provided by a server 106 and as a server 106 providing access to hosted resources for other client devices 102A-102N. The networks 104, 104′ may be generally referred to as a network 104. The networks 104 may be configured in any combination of wired and wireless networks.

A server 106 may be any server type such as, for example: a file server; an application server; a web server; a proxy server; an appliance; a network appliance; a gateway; an application gateway; a gateway server; a virtualization server; a deployment server; a Secure Sockets Layer Virtual Private Network (SSL VPN) server; a firewall; a web server; a server executing an active directory; a cloud server; or a server executing an application acceleration program that provides firewall functionality, application functionality, or load balancing functionality.

A server 106 may execute, operate or otherwise provide an application that may be any one of the following: software; a program; executable instructions; a virtual machine; a hypervisor; a web browser; a web-based client; a client-server application; a thin-client computing client; an ActiveX control; a Java applet; software related to voice over internet protocol (VoIP) communications like a soft IP telephone; an application for streaming video and/or audio; an application for facilitating real-time-data communications; a HTTP client; a FTP client; an Oscar client; a Telnet client; or any other set of executable instructions.

In some embodiments, a server 106 may execute a remote presentation services program or other program that uses a thin-client or a remote-display protocol to capture display output generated by an application executing on a server 106 and transmit the application display output to a client device 102.

In yet other embodiments, a server 106 may execute a virtual machine providing, to a user of a client device 102, access to a computing environment. The client device 102 may be a virtual machine. The virtual machine may be managed by, for example, a hypervisor, a virtual machine manager (VMM), or any other hardware virtualization technique within the server 106.

In some embodiments, the network 104 may be: a local-area network (LAN); a metropolitan area network (MAN); a wide area network (WAN); a primary public network 104; and a primary private network 104. Additional embodiments may include a network 104 of mobile telephone networks that use various protocols to communicate among mobile devices. For short range communications within a wireless local-area network (WLAN), the protocols may include 802.11, Bluetooth, and Near Field Communication (NFC).

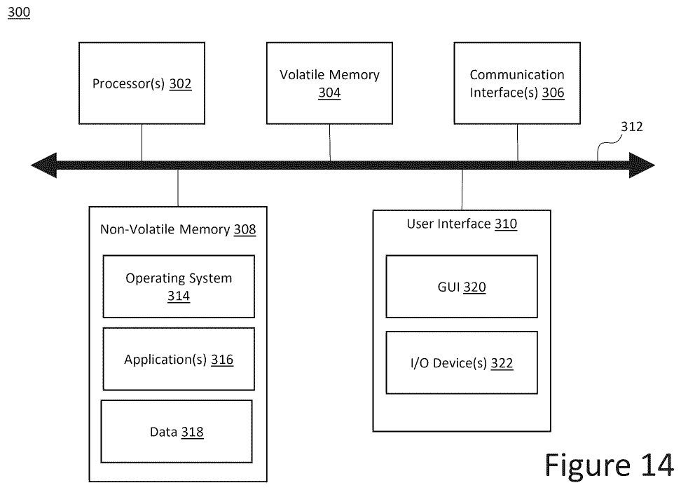

Elements of the described solution may be embodied in a computing system, such as that shown in FIG. 14 in which a computing device 300 may include one or more processors 302, volatile memory 304 (e.g., RAM), non-volatile memory 308 (e.g., one or more hard disk drives (HDDs) or other magnetic or optical storage media, one or more solid state drives (SSDs) such as a flash drive or other solid state storage media, one or more hybrid magnetic and solid state drives, and/or one or more virtual storage volumes, such as a cloud storage, or a combination of such physical storage volumes and virtual storage volumes or arrays thereof), user interface (UI) 310, one or more communications interfaces 306, and communication bus 312. User interface 310 may include graphical user interface (GUI) 320 (e.g., a touchscreen, a display, etc.) and one or more input/output (I/O) devices 322 (e.g., a mouse, a keyboard, etc.). Non-volatile memory 308 stores operating system 314, one or more applications 316, and data 318 such that, for example, computer instructions of operating system 314 and/or applications 316 are executed by processor(s) 302 out of volatile memory 304. Data may be entered using an input device of GUI 320 or received from I/O device(s) 322. Various elements of computer 300 may communicate via communication bus 312. Computer 300 as shown in FIG. 11 is shown merely as an example, as clients, servers and/or appliances and may be implemented by any computing or processing environment and with any type of machine or set of machines that may have suitable hardware and/or software capable of operating as described herein.

Processor(s) 302 may be implemented by one or more programmable processors executing one or more computer programs to perform the functions of the system. As used herein, the term “processor” describes an electronic circuit that performs a function, an operation, or a sequence of operations. The function, operation, or sequence of operations may be hard coded into the electronic circuit or soft coded by way of instructions held in a memory device. A “processor” may perform the function, operation, or sequence of operations using digital values or using analog signals. In some embodiments, the “processor” can be embodied in one or more application specific integrated circuits (ASICs), microprocessors, digital signal processors, microcontrollers, field programmable gate arrays (FPGAs), programmable logic arrays (PLAs), multi-core processors, or general-purpose computers with associated memory. The “processor” may be analog, digital or mixed-signal. In some embodiments, the “processor” may be one or more physical processors or one or more “virtual” (e.g., remotely located or “cloud”) processors.

Communications interfaces 306 may include one or more interfaces to enable computer 300 to access a computer network such as a LAN, a WAN, or the Internet through a variety of wired and/or wireless or cellular connections.

In described embodiments, a first computing device 300 may execute an application on behalf of a user of a client computing device (e.g., a client), may execute a virtual machine, which provides an execution session within which applications execute on behalf of a user or a client computing device (e.g., a client), such as a hosted desktop session, may execute a terminal services session to provide a hosted desktop environment, or may provide access to a computing environment including one or more of: one or more applications, one or more desktop applications, and one or more desktop sessions in which one or more applications may execute.

As will be appreciated by one of skill in the art upon reading the following disclosure, various aspects described herein may be embodied as a system, a device, a method or a computer program product (e.g., a non-transitory computer-readable medium having computer executable instruction for performing the noted operations or steps). Accordingly, those aspects may take the form of an entirely hardware embodiment, an entirely software embodiment, or an embodiment combining software and hardware aspects. Furthermore, such aspects may take the form of a computer program product stored by one or more computer-readable storage media having computer-readable program code, or instructions, embodied in or on the storage media. Any suitable computer readable storage media may be utilized, including hard disks, CD-ROMs, optical storage devices, magnetic storage devices, and/or any combination thereof.

The foregoing drawings show some of the processing associated according to several embodiments of this disclosure. In this regard, each drawing or block within a flow diagram of the drawings represents a process associated with embodiments of the method described. It should also be noted that in some alternative implementations, the acts noted in the drawings or blocks may occur out of the order noted in the figure or, for example, may in fact be executed substantially concurrently or in the reverse order, depending upon the act involved. Also, one of ordinary skill in the art will recognize that additional blocks that describe the processing may be added.

What is claimed is:

1. A system, comprising:
a memory; and
a processor coupled to the memory and configured to provide a data collaboration infrastructure, having:
a data handling service for reading legacy data from and writing legacy data to a plurality of disparate information systems and for transforming and processing legacy data using a common universal data record (UDR) format;
a universal display interface (UDI) service configured to:
display a UDI at remote clients, wherein the UDI includes a primary interactive diagram; and
overlay case-specific graphical information onto the primary interactive diagram, wherein the graphical information is rendered from data obtained from at least one disparate information system; and a collaboration service configured to:
create active capture recordings from within the UDI at the remote clients, wherein the active capture recordings include screen activity, audio, and video footage; and
share active capture recordings with other remote clients in a secure collaboration interface within the UDI.

2. The system of claim 1, wherein reading legacy data from and writing legacy data to the plurality of disparate information systems utilizes robotic process automation to interact with user interfaces of the plurality of disparate information systems.

3. The system of claim 1, wherein reading legacy data from and writing legacy data to the plurality of disparate information systems utilizes application programming interfaces.

4. The system of claim 1, wherein plurality of disparate information systems utilize different commands and file systems to access and store data.

5. The system of claim 1, wherein the UDR format includes a system agnostic file type for similar types of data.

6. The system of claim 5, wherein the UDR format further includes a system agnostic naming convention.

7. The system of claim 1, wherein the legacy data includes dental image data and dental text data.

8. The system of claim 7, wherein the primary interactive diagram includes a generic chart of a set of teeth.

9. The system of claim 8, wherein the overlay depicts patient specific dental work rendered on the set of teeth.

10. The system of claim 9, wherein the UDI further includes a media file viewer for viewing dental images of a selected patient.

11. The system of claim 10, wherein the UDI further includes a notes interface for viewing and creating patient specific notes.

12. The system of claim 11, further comprising a secure script artificial intelligence (S2AI) service configured to securely transcribe and store natural language inputs within the notes interface.

13. The system of claim 12, wherein the S2AI service includes a generative AI enhancement service that processes natural language inputs.

14. The system of claim 13, wherein the generative AI enhancement service converts natural language inputs into formatted medical notes.

15. The system of claim 14, wherein the collaboration interface includes a facility for a first user to create a collaboration with a second user, and a content window for viewing and sharing messages with the second user.

16. The system of claim 15, wherein the content widow is configured to send and display active capture recordings.

17. The system of claim 16, wherein the collaboration service includes annotation tools for annotating displays within the UDI.

18. The system of claim 7, wherein the UDI further includes a secondary interactive diagram that comprises a generic periodontic chart of a set of teeth.

19. The system of claim 18, wherein the UDI service is configured to overlay patient specific periodontic information onto the secondary interactive diagram, wherein the graphical information is rendered from data obtained from at least one disparate information system.

20. A method, comprising:
rendering a universal display interface (UDI) at a remote client, wherein the UDI includes a primary interactive diagram;
reading legacy data from at least one of a plurality of disparate information systems and transforming the legacy data into a common universal data record (UDR) format;
overlaying patient specific graphical information onto the primary interactive diagram, wherein the graphical information is rendered from legacy data obtained from the at least one of the plurality of disparate information system;
providing a facility for creating an active capture recording from within the UDI, wherein the active capture recording includes screen activity, audio, and video footage; and
providing a collaboration interface within the UDI, and securely sharing a collaboration message with a second remote client, wherein the collaboration message includes the active capture recording.
